(19) United States
(12) Patent Application Publication
(10) Pub. No.: US 2025/0271253 A1
(43) Pub. Date: Aug. 28, 2025

(54) SELF-MIXING INTERFEROMETRY SENSOR MODULE, ELECTRONIC DEVICE AND METHOD OF DETECTING MOVEMENT

(71) Applicant: ams International AG, Jona (CH)

(72) Inventors: Bobby DANIEL, Eindhoven (NL); Ferran SUAREZ, Rüschlikon (CH); Daniel NAJER, Rotkreuz (CH)

(21) Appl. No.: 18/865,605

(22) PCT Filed: May 10, 2023
(86) PCT No.: PCT/EP2023/062412

§ 371 Date: Nov. 13, 2024

Related U.S. Application Data
(60) Provisional application No. 63/344,873, filed on May 23, 2022.

(51) Int. Cl. G01B 9/02; A61B 5/00; A61B 5/024; G01B 9/02055

(52) U.S. Cl. CPC G01B 9/02092; A61B 5/02427; A61B 5/681; A61B 5/7214; G01B 9/02076

(57) ABSTRACT

A self-mixing interferometry sensor module includes at least two light emitters of the same type, a detector unit and an electronic processing unit. Each light emitter is operable to emit coherent electromagnetic radiation out of the sensor module and undergo self-mixing interference (SMI) caused by reflections of the emitted electromagnetic radiation from an external object outside the sensor module. The detector unit is operable to generate output signals indicative of the SMI of the light emitters, respectively. The electronic processing unit is operable to generate a difference signal from the output signals indicative of a movement of the external object.

This disclosure relates to a self-mixing interferometry sensor module, an electronic device and to a method of detecting movements.

Electronic devices such as smartphones, watches, and other wearable devices include an increasing number of sensors for sensing and/or monitoring physiological parameters of a user. For example, such wearable electronic devices are able to measure or monitor parameters such as heart rate, blood oxygen saturation, blood pressure, sleep cycles, body temperature, etc.

The electronic devices comprise sensor modules that derive a physiological state of the user (e.g. blood oxygen saturation, heart rate, etc.) by applying a stimulus, such as light, and detecting a response from the interaction of the stimulus with the body. For example, some sensor modules comprise light-emitting diodes (LEDs) to emit light, such as infrared light, into a blood vessel and detect the response of the light after it interacts with the blood. In such cases, one or more conditions of the blood (e.g. flow rate, oxygen content, etc.) may affect the transmitted light, which can be used to derive a physiological parameter such as blood oxygen saturation.

Self-mixing interference relates to light waves emitted from the laser being reflected back into a resonant cavity of a light emitter, such as a laser, by an external object and interfering with the light waves in the resonant cavity. Since the feedback light carries information of external objects, output signals of the light emitter can be modulated by the external object, so that a physical parameter of the target can be measured by using this characteristic. An SMI interferometer, or self-mixing interferometry sensor, SMI sensor hereinafter, may only need one optical path, so it offers a simple and compact structure, combined with easy collimation, high sensitivity, and convenient signal detection, so that the self-mixing interference has been widely used to measure displacement, vibration, topography, acceleration, small angle, etc.

In recent years, however, self-mixing interference has become available for sensing and/or monitoring physiological parameters using wearable electronic devices. SMI sensor technology can measure displacements of a target with a resolution in the order of 500 nm or lower, depending on the wavelength provided by light emitters. The technology also allows to measure the velocity of a target with high precision. This property has made it currently a hot topic in the field of vital signs monitoring, where it can (among other things) measure the pulse micro-movements to measure the heart rate, or measure the speed of flow of blood particles in vessels close to the skin surface.

In many cases, the accuracy or quality of measurements from these modules depends on the light penetrating the skin and interacting with one or more blood vessels. Therefore, these sensors may be sensitive to noise (e.g. respiratory movements, muscle contractions, electronic noise, etc.) and require robust circuitry to detect and/or process the signals received from the user's body. Another significant challenge in implementing it in a wearable device (like a smart watch) is that any unwanted motion of the body part (wrist) or tissue, relative to the SMI-sensor (e.g. in the watch), results in noise superimposed on the SMI output signal.

A method of processing the SMI output signal (which may comprise the measured laser power) may involve a fast Fourier transform, FFT, to extract a dominant frequency from the output signals. (There are other algorithms which may also be used on the FFT to extract a quantity which is proportional to the velocity of movement. For example, a ratio of moments of the FFT function also gives a quantity with the dimension of frequency and is proportional to the velocity of a target's movement.) This dominant frequency, denoted f, may then be related to the velocity v of movement of the target as f=2*v/wavelength. For example, when there is movement of a whole hand (or the tissue itself in which the vessel is embedded), this adds an additional velocity component and the dominant frequency is shifted with respect to no movement. If the movement is random, however, it is not possible to distinguish the contribution of the pulsation (or blood flow) velocity from that of the unwanted movement.

An object to be achieved is to provide a sensor module for electronic devices that overcomes the aforementioned limitations of existing solutions. A further object is to provide a wearable electronic device comprising such a sensor module and a method of detecting movements.

These objects are achieved with the subject-matter of the independent claims. Further developments and embodiments are described in dependent claims.

### SUMMARY OF THE INVENTION

The following relates to an improved concept in the field of optical sensing. One aspect relates to a self-mixing interferometry sensor module which can be used for detecting movements of an object external to the module. For example, the module allows for sensing and/or monitoring of one or more physiological parameters of a user. Furthermore, the sensor module can be integrated into a wearable electronic device to measure or monitor parameters such as heart rate, blood oxygen saturation, blood pressure, sleep cycles, body temperature, etc.

Another aspect relates to the use of two essentially identical light emitters, e.g. VCSEL laser diodes, which are rigidly connected to each other and separated by a distance, which may be optimized for a differential measurement. For example, one signal component may be significantly different in terms of the output signals provided by the light emitters, but a motion related artefact noise component can be significantly similar. The proposed concept may be extended to include an array of light emitters.

In at least one embodiment, a self-mixing interferometry sensor module comprises at least two light emitters of the same type, a detector unit and an electronic processing unit.

Each light emitter is operable to emit coherent electromagnetic radiation out of the sensor module, e.g. towards an external object. Furthermore, the light emitters are configured to undergo self-mixing interference, SMI, which may be caused by reflections of the emitted electromagnetic radiation from an external object outside the sensor module.

The detector unit is operable to generate output signals which are indicative of the SMI of the light emitters, respectively.

The electronic processing unit is operable to generate a difference signal from the output signals generated by the detector unit. In fact, the difference signal is indicative of a movement of the external object. (Hereinafter, the difference signal generated may not be a subtraction of the raw SMI output signals, but a subtraction of parameters extracted from the outputs signals, e.g. velocity or a dominant frequency from FFT).

For example, the light emitters are arranged for enabling self-mixing interference, and typically comprise a cavity resonator, into which at least a fraction of the light emitted by the light emitters can be reflected, or backscattered, from the external object outside the module. For example, the light emitters are implemented as a laser diode and comprise a laser cavity. The light emitters are configured to emit coherent light, e.g. in an infrared (IR), visible or ultraviolet (UV) range of the electromagnetic spectrum, out of the sensor module. For example, the light emitters are configured to generate a continuous emission or to emit light in a pulsed fashion, the latter potentially aiding in achieving an overall reduction in power consumption.

Upon the aforementioned back-injection of the emitted light into the cavity and due to a movement of the object outside the module the light is reflected off, the light emitters are configured to undergo self-mixing interference. The SMI alters a property, e.g. a wavelength, of the light within or emitted from the laser cavity, e.g. it causes a modulation in an amplitude and/or frequency of the emitted light, hence generating periodic fringes in the signal. More accurately, SMI modulates the optical power (which is usually observed by measuring the optical power or the biasing voltage). In turn, the modulation causes a change in an electronic property of the light emitter. For example, a diode current and/or voltage is likewise modulated. When no target is present outside the module in the field of emission of the light source so as to intercept and reflect light of the latter, no self-mixing interference occurs within the light emitter. The term light emitters of the “same type” indicates that the light emitters are essentially the same, e.g. two copies of the same design.

As discussed above, SMI eventually alters a property of the light emitters. This property is indirectly measured by means of the detector unit, which generates the output signals as a function of said property, or change in said property. The output signals may be a measured current or voltage, for example. Thus, the detector unit may comprise means, e.g. active or passive circuitry, to measure said change as an electronic property.

Furthermore, the electronic processing unit receives the output signals and, for each pair of light emitters, generates a difference signal from the output signals, i.e. a difference based on extracted parameters. On the one hand, the difference signal is a function of a movement of the external object. On the other hand, the process of taking a difference of the output signal inherently accounts for noise. As the light emitters are of the same type and arranged together in the same module, it is safe to assume that the emitters will be affected by noise in a similar fashion. Thus, the difference signal may have a lower impact of noise, while containing the information on movement of the individual output signals. In effect, the signal to noise ratio (short: SNR) can be increased.

A detected change in the difference signal, i.e. in the output signals, or detected electronic property, depends on the movement of the external object. The sensor module may comprise further circuitry, or the functionality of the electronic processing unit may be extended, for deriving from the change in difference signal a movement of the external object, e.g. in terms of a change in position, direction and/or speed. Based on the determined movement, the sensor module further comprises means to generate a processed output signal that contains information of the determined movement.

For example, the movement is a movement perpendicular or parallel to an emission direction of the light emitters. The processed output signal comprises information about the movement, e.g. it comprises information of speed, duration and/or direction of the movement as well as a distance of the object, e.g. measured in an emission direction of the light emitters.

The improved concept provides motion artefact reduction and SNR improvement by separating signal and noise into different regimes (differential mode and common mode, respectively). No separate motion sensor (such as an accelerometer) is required to help in removing motion artefacts. A comparably simple design change and hardware addition may suffice to integrate two or more light emitters in the sensor module, e.g. identical VCSELs instead of one. Simple subtraction is sufficient to yield an increase in SNR, as opposed to complex computation seen in the prior art.

In at least one embodiment, the light emitters, detector unit and the electronic processing unit form an integrated semiconductor device, such as a CMOS integrated circuit device, on a common substrate. In particular, a sensor module according to the improved concept can be free of any dedicated photodetectors for sensing reflected or backscattered light. In addition, or alternatively, the sensor module comprises a sensor package into which the light emitters, detector unit and the electronic processing unit, or the integrated semiconductor device formed by the light emitters, detector unit and the electronic processing unit, are integrated.

A wearable electronic device may use such a self-mixing interferometry sensor module as part of detecting a displacement, distance, motion, speed, or velocity of an object outside the module, e.g. a gesture at a distance from the sensor module or a movement with respect to an input surface of the module, i.e. a so-called swiping or tapping movement. Hereinafter, all such possibly measured parameters will be referred to simply as “movement”. Specifically, such a self-mixing interferometry sensor module can be used with a wide range of consumer and other electronic devices. For example, the movement can be related to displacements of a target within a human body to sense and/or monitor physiological parameters, e.g. to measure the pulse micro-movements to measure the heart rate, or measure the speed of flow of blood particles in vessels close to the skin surface.

In at least one embodiment, the light emitters essentially have the same configuration, including essentially the same emission wavelengths. In addition to using light emitters of the same type, i.e. copies of the same design, reducing the impact of noise is further supported by configuring the light emitters in the same way. Such configuration may include driving current, so that the light emitters emit light with essentially the same output power or intensity. Furthermore, the light emitters may be set or driven to emit the coherent electromagnetic radiation with essentially the same wavelength or range of wavelengths.

In at least one embodiment, the light emitters are rigidly coupled to each other. The term “rigid coupling” indicates that a distance between any two light emitters is essentially constant, e.g. on a scale of operation of the sensor module. In other words, the light emitters have a constant relationship in terms of their distance. This relationship may be constant under changing temperature or under mechanical stress acting on the sensor module. As a consequence the light emitters have a determined, or possibly identical, direction of emission. Furthermore, any pair of light emitters can be characterized such that under defined conditions (e.g. a condition without any movement) their output signals allow to determine a reference difference signal.

In at least one embodiment, the light emitters are arranged in pairs at a distance from each other. The distance is chosen such that the output signals indicative of the SMI of the pair of light emitters differ by a pre-determined amount.

In order to generate a difference signal at least two light emitters are arranged in the sensor module. However, any desired number of light emitters may be integrated into the sensor module, so that any one of these may undergo SMI and corresponding output signals are generated by the detector unit indicative of the SMI, respectively. Difference signals can be generated from any pair of output signals to result in a corresponding difference signal. Thus, the electronic processing unit is operable to generate difference signals from the output signals in a pairwise manner.

The distance between any pair of light emitters, in case of only two emitters or an array of light emitters, may account for said pre-determined amount. However, the actual amount highly depends on the desired application and may be subject to experimentation and design choices. When choosing the distance, it may be helpful to consider the following guideline.

Consider a pair of two light emitters. The two light emitters may be separated by a distance such that the signal component of their respective output signals is significantly different in the two, but the motion artefact noise component is essentially similar. For example, in terms of signal strength the two output signals may differ by up to a factor of 10. The following provides a guideline to determine a minimum and/or maximum distance between light emitters.

Consider, for example, an array of light emitters (e.g. VCSELs with the same epi design, same substrate wafer). The distances between individual lasers of the array may be in the order of 250 μm pitch. Individual spots on the target may be separated with optics or meta-optics. Thus, a minimum distance can be determined by the fabrication-typical pitch. A maximum distance may be determined on the desired illumination on the target side, via e.g. the comparison of having the laser beam hitting the capillary vs outside the capillary. The bigger radius of the capillary depends on the aorta (some 2.5 mm, for example). The distance between light emitters may be in this range so as to separate the capillary. Thus, the lasers of the array may be separated between hundreds of um to a few mm. Note that the laser spots can be separated further by the optics and the distance from the sensor to the capillary (some 5 mm, for example). If the laser beams have to be parallel for simplicity (incident angle can be accounted for), the distance between the light emitters may be in the range of some mm, so that one emitter is on the capillary and the other out of it. That is to say, the other one is e.g. displaced with respect to the latter.

As a further guideline, the distance between the light emitters, e.g. in a pair, may be set to enable a noise component to become common mode and a signal component to be differential mode in the corresponding output signals. Thus, the difference signal may cut down the motion artifacts significantly while reducing the signal only slightly. The signal-to-noise ratio can be greatly improved.

For example, in a wrist-based wearable electronic device for heart rate monitoring, the size of blood vessels and their separation on the wrist will be used to calculate the separation. The positioning of the light emitters, and their distance, should provide a high probability that one of the two sensors has a significantly greater proximity to the source of the signal, i.e. pulsating vessel. The distance, however, should be close enough that the unwanted motion between the wrist and the sensors is largely identical for the two light emitters.

In at least one embodiment, the sensor module comprises an array of the light emitters. The electronic processing unit is operable to generate difference signals from the output signals of pairs of the light emitters.

In at least one embodiment, the light emitters are arranged in parallel such that the light emitters have the same direction of emission. This way the light emitters emit coherent light towards the same target but with an offset distance.

In at least one embodiment, the light emitters comprise semiconductor laser diodes and/or resonant cavity light emitting devices. These devices feature coherent emission to generate SMI fringes.

A resonant cavity light emitting device can be considered a semiconductor device, similar to a light emitting diode, which is operable to emit light based on a resonance process. In this process, the resonant cavity light emitting device may directly convert electrical energy into light, e.g. when pumped directly with an electrical current to create amplified spontaneous emission. However, instead of producing stimulated emission only spontaneous emission may result, e.g. spontaneous emission perpendicular to a surface of the semiconductor is amplified.

In at least one embodiment, the light emitters comprise vertical cavity surface emitting laser, VCSEL, diodes. VCSELs are an example of a resonant cavity light emitting device and feature a beam emission that is perpendicular to a main extension plane of a top surface of the VCSEL. The VCSEL diode can be formed from semiconductor layers on a substrate, wherein the semiconductor layers comprise two distributed Bragg reflectors (DBRs) enclosing active region layers in between and thus forming a cavity. VCSELs and their principle of operation are a well-known concept and are not further detailed in this disclosure. For example, the VCSEL diode is configured to have an emission wavelength of 940 nm, 850 nm, or another natural wavelength. The VCSEL diode can be configured to emit coherent laser light when forward biased, for instance.

In at least one embodiment, the electronic processing unit is operable to conduct a fast Fourier transformation, FFT, on the output signals to extract a dominant frequency. The difference signal is then generated as a function of the dominant frequency.

As discussed above, in SMI a fraction of the coherent light emitted by the light emitters may be reflected by an external object and eventually is re-injected into the emitter, e.g. a laser cavity. A movement of the target causes a modulation in the detected output signals. For example, the power emitted by the light emitter is in fact modulated both in amplitude (AM) and in frequency (FM), generating a fringes in the interferometric output signal. Thus, the output signals are affected by frequency and may be periodic functions of the phase of the back-scattered field. Thus, the output signals can be treated with (fast) Fourier transformation to extract characteristic frequencies, e.g. a dominant frequency.

A possible processing may involve fast Fourier transformation on the output signals as a first step. Then, one or more dominant frequencies may be extracted as a result of FFT. The difference signal may be generated from the FFT result, e.g. subtracting FFT intermediate signals as a whole. Alternatively, the difference or dominant frequencies can be determined. Finally, the extracted difference signal can be related to movement or velocity of the external target.

In at least one embodiment, the detector unit is operable to detect a junction voltage of the light emitters, respectively. The output signals are then generated as a function of said junction voltages, respectively. Junction voltage is one possible electronic property of the light emitters which may change as a result of SMI. For example, the detector unit comprises one or more voltage meters to detect the junction voltage(s).

In at least one embodiment, the detector unit is operable to detect an optical power output of the light emitters, respectively. The output signals are then generated as a function of said optical power outputs, respectively. Optical power is another possible property of the light emitters which may change as a result of SMI. For example, the detector unit comprises one or more photodetectors, such as a photodiode, to detect optical power outputs.

In at least one embodiment, a wearable electronic device comprises a self-mixing interferometry sensor module according to one of the aforementioned aspects. Furthermore, it comprises a housing comprising the sensor module and a support surface. The support surface is to be arranged on the skin of a user. The housing is configured to position the light emitters at a distance from the skin of the user.

For example, wearable electronic devices are considered to be any device which by their appearance are wearable and/or which are designed to be worn on the body. A wearable electronic device may be required to be worn to function, e.g. may be conceptually linked to the wearer's body. Some wearable electronic devices may require the user interface to be present and available all the time, others may require no input (such as a wrist unit or chest belt of a heart-rate monitor). Wearable electronic devices may include a smartwatch or fitness tracker, and the like.

In at least one embodiment, the wearable electronic device comprises a processing unit which is coupled to the sensor module. The processing unit is configured to receive the difference signals from the sensor module and determine a movement of the external object outside the sensor module. Furthermore, the processing unit generates a processed output signal that comprises information of the determined movement.

The processing unit can be a central processing unit, CPU, of the wearable electronic device, or a system-on-a-chip, SOC, that is dedicated to process output signals of the light emitters, for instance. The output signal contains information about a detected movement, e.g. a direction, distance and/or speed of the movement of an external object, e.g. located below the skin of the user.

In at least one embodiment, the light emitters are arranged in the housing, such that the direction of emission of the light emitters is essentially perpendicular to the support surface. The wearable electronic device, including the sensor module, may rest on the skin with the support surface down. In this configuration the light emitters are separated by a distance with respect to each other, but essentially have the same distance with respect to the skin (and support surface for that matter). Thus, a difference in signal strength of the output signals may be due to different reflection or scattering at the external object. For example, a blood vessel may be spherical or ellipsoidal on shape. Thus, the coherent light of one light emitter may be reflected or scattered from a different relative distance than the coherent light of a neighboring light emitter.

In at least one embodiment, the light emitters are arranged in the housing, such that the direction of emission of the light emitters is tilted with respect to the support surface. The wearable electronic device, including the sensor module, may rest on the skin with the support surface down. In this configuration the light emitters are separated by a distance with respect to each other, and are tilted with respect to the support surface. Thus, the light emitters by design have a different distance with respect to the skin (and the support surface for that matter). A difference in signal strength of the output signals may be due to different reflection or scattering at the external object. However, due to the tilted arrangement light paths towards the object may be affected by movement of the sensor module in the same way for both light emitters. This allows for increasing spatial resolution further, and signal differences may be more pronounced, while noise remains at the same common mode level.

Further embodiments of the electronic device become apparent to the skilled reader from the aforementioned embodiments of the self-mixing interferometry sensor module, and vice-versa.

Furthermore, a method of detecting movements is provided, comprising at least the following steps. One step includes emitting, by means of two light emitters of the same type, coherent electromagnetic radiation out of a sensor module. Another step includes inducing, within the light emitters, self-mixing interference, SMI, caused by reflections of, or scattering by, the emitted electromagnetic radiation from an object external to the sensor module. Another step includes generating output signals indicative of the SMI of the light emitters, respectively. Another step includes generating a difference signal from the output signals indicative of a movement of the external object.

These steps can be complemented by further procedural steps, such as determining from the difference signal a movement of the external object, and generating a processed output signal that comprises information of the determined movement.

Further embodiments of the method become apparent to the skilled reader from the aforementioned embodiments of the self-mixing interferometry sensor module and of the wearable electronic device, and vice-versa.

The following description of figures may further illustrate and explain aspects of the self-mixing interferometry sensor module, wearable electronic device and the method of detecting movements. Components and parts of the self-mixing interferometry sensor that are functionally identical or have an identical effect are denoted by identical reference symbols. Identical or effectively identical components and parts might be described only with respect to the figures where they occur first. Their description is not necessarily repeated in successive figures.

In the figures:

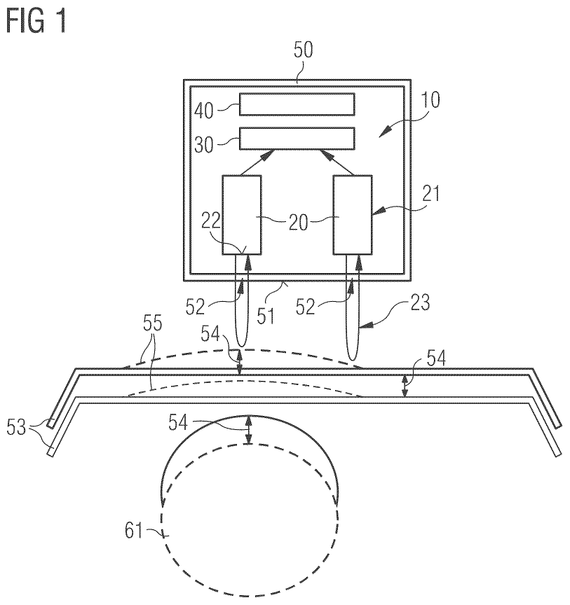

FIG. 1 shows an exemplary embodiment of a self-mixing interferometry sensor module for a wearable electronic device,

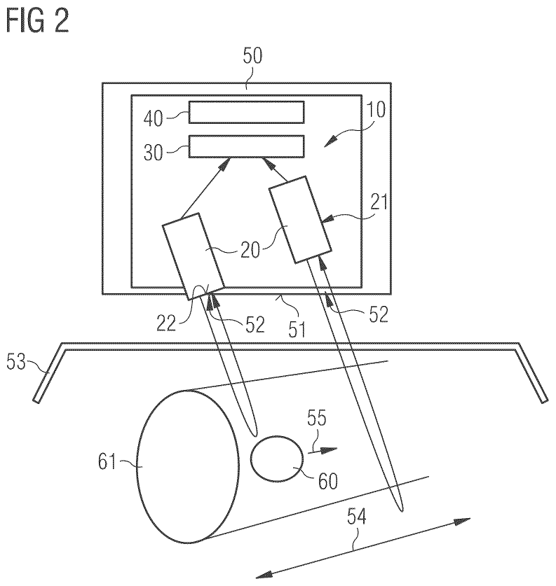

FIG. 2 shows another exemplary embodiment of a self-mixing interferometry sensor module for a wearable electronic device, and

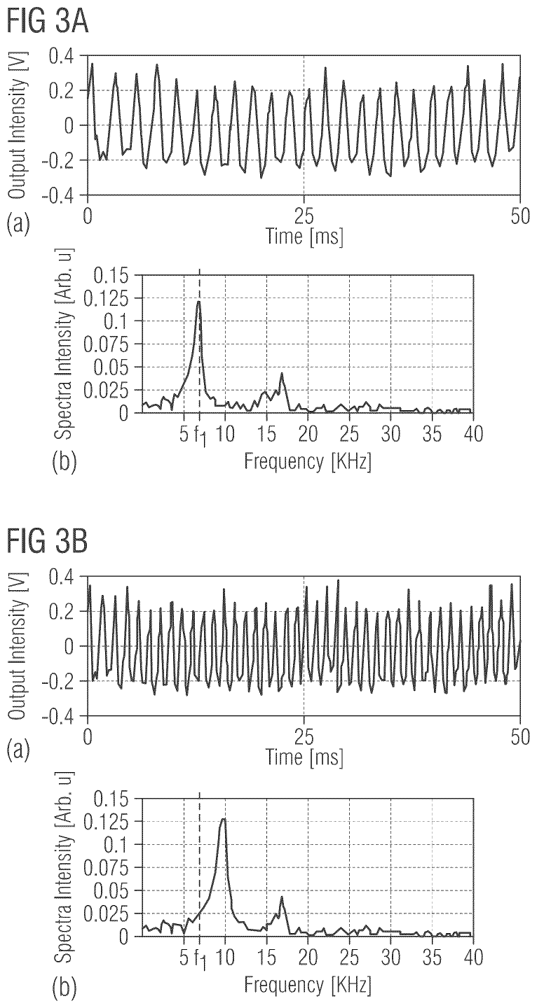

FIGS. 3A, 3B show exemplary measurements conducted with a self-mixing interferometry sensor module for a wearable electronic device.

The proposed self-mixing interferometry sensor module can be used in various electronic devices, including wearable electronic devices, such as smartwatches or fitness trackers, and the like. For example, the sensor module enables an electronic device (wearable or not) to derive a physiological state of the user (e.g. blood oxygen saturation, heart rate, etc.) by applying light as a stimulus, and detecting a response from the interaction of the stimulus with the body. In the following, two possible examples are disclosed which can be considered representative of the various possible applications. The examples relate to pulse monitoring and blood flow measurements.

FIG. 1 shows an exemplary embodiment of a self-mixing interferometry sensor module for a wearable electronic device. The drawing shows a wearable electronic device, e.g. a smartwatch, comprising a sensor module 10. The sensor module is integrated into and electrically connected to the wearable electronic device. In fact, the wearable electronic device comprises a housing 50 with a support surface 51. The sensor module is placed or mounted in the housing.

The sensor module 10 further comprises two light emitters 20, a detector unit 30 and an electronic processing unit 40. The sensor module can be implemented as a sensor package, into which the light emitters, detector unit and the electronic processing unit, or the integrated semiconductor device formed by the light emitters, detector unit and the electronic processing unit, are integrated. For example, the detector unit and the electronic processing unit form an integrated semiconductor device, such as a CMOS integrated circuit device, on a common substrate. The light emitters can either be integrated into the integrated semiconductor device or be electrically connected to the integrated semiconductor device as external components.

The light emitters 20 are implemented being of the same type, i.e. copies of the same design. In this embodiment, the two light emitters are vertical cavity surface emitting laser, or VCSEL, diodes. VCSELs are an example of resonant cavity light emitting devices. The light emitters comprise semiconductor layers with distributed Bragg reflectors (not shown) which enclose active region layers in between, thus forming a cavity 21. The VCSELs feature a beam emission of coherent electromagnetic radiation that is perpendicular to a main extension plane of a top surface 22 of the VCSEL. For example, the VCSEL diodes are configured to have an emission wavelength in the infrared range, e.g. at 940 nm or 850 nm.

The sensor module 10 may comprise a laser driver integrated into the integrated semiconductor device as a means to drive the light emitters. The number of light emitters which are implemented may only be restricted by the desired application. The two emitters discussed herein should be considered an example, rather than any restriction of the proposed concept.

The light emitters 20 are arranged in the housing 50 and parallel with respect to each other, such that the direction of emission 23 of the light emitters is essentially perpendicular to the support surface 51. Furthermore, the housing comprises apertures 52 arranged in the support surface in front of the light emitters, respectively. The light emitters 20 are rigidly coupled to the sensor module 10 and are separated by a distance. The distance is set such that the signal component of their respective output signals can be significantly different in the two light emitters. As a possible guideline, the distance between light emitters, e.g. in a pair, may be set to enable a noise component to become common mode and a signal component to be differential mode in the corresponding output signals.

The detector unit 30 is shown as a schematic building block. The detector unit comprises means, e.g. active or passive circuitry, to measure an optical or electronic property of the light emitters 20. For example, the detector unit comprises a current or voltage meter to detect a junction voltage of the light emitters, respectively. Junction voltage is one possible electronic property of the light emitters and may change as a result of self-mixing interference. In addition, or alternatively, the detector unit comprises one or more photodetectors such as a photodiode to detect an optical power output of the light emitters, respectively. The optical power output is a possible optical property of the light emitters and may change as a result of self-mixing interference. In some embodiments, the photodetectors can be integrated on the epitaxy of the light emitters 20.

The lasers may have optics (not shown in the figure), of integrated (meta-lens, etched in the substrate) or separated (physical individual lens: micro-optics or lenses) kind, or a common lens or an array of micro-lenses. Focusing is for example needed for sufficient SMI signal strength in blood-flow velocity measurements.

The electronic processing unit 40 constitutes a functional unit of the sensor module, which conducts a number of (pre-)processing steps. Its functionality will be discussed in further detail below. These steps include conducting of a fast Fourier transformation on output signals of the light emitter and subtracting of signals, e.g. of the result of FFT, to generate difference signals. For example, the electronic processing unit comprises a microprocessor or ASIC.

The wearable electronic device comprises additional components (not shown), such as a processing unit, to receive the difference signals from the sensor module and determine a movement of an external object outside the sensor module. The processing unit can be a central processing unit, CPU, of the wearable electronic device, microprocessor, or a system-on-a-chip, SOC, which is dedicated to process output signals of the light emitters 20, for instance. As will be discussed in further detail below, the output signals, or difference signals, contain information about a detected movement, e.g. a direction, distance and/or speed of the movement of an external object, e.g. located below the user's skin 53.

In operation, the wearable electronic device is placed on a user's skin 53 with the support surface 51 of the housing 50 facing down. This way, the apertures 52 in front of the light emitters 20 face the user's skin 53 and provide respective openings to irradiate the skin by means of the light emitters. The light emitters emit coherent light, e.g. in an infrared (IR), visible or ultraviolet (UV) range of the electromagnetic spectrum, out of the sensor module and, via the apertures, towards the skin. For example, the light emitters generate a continuous emission or emit light in a pulsed fashion, wherein the latter potentially aids in achieving an overall reduction in power consumption.

In this exemplary application (pulse monitoring) the skin 53 constitutes the moving external object, or target. Output signals provided by the light emitters 20 are indicative of a displacement of the skin. The displacement can be retrieved using the output signals, or difference signals, as outlined in the method of detecting a movement below. Basically, the output signals generated by the light emitters due to self-mixing interference, SMI, are affected by a movement of the skin (surface) along the optical paths of the light emitters, as indicated by arrows 54 in the drawing. This is caused by different placement of the electronic device on the skin, or relative movement, and typically leads to a noise component. This way, a relative distance between light emitters 20 and blood vessels 60 may be different for each light emitter (see cross-section 61 of a skin surface in the drawing).

Coherent light, emitted by the light emitters, strikes the skin 53. A fraction of the light is reflected off the surface of the skin and scattered back towards the light emitters 20. Eventually, the reflected light enters the sensor module via the apertures 52 and is injected back into the cavities 21 of the light emitters. In the laser cavity the injected light leads interferes with the coherent light which is just being generated by the light emitters. As a result, the light emitters undergo self-mixing interference, or SMI for short. Furthermore, SMI alters the lasing process in the cavity and results in a change in optical or electronic properties, e.g. a wavelength, of the light within or emitted from the laser cavity. For example, SMI causes a modulation in an amplitude and/or frequency of the emitted light, hence generating a periodic fringing signal. In turn, the modulation causes a change in an electronic property of the light emitters. For example, a diode current and/or voltage are likewise modulated. Either voltage or optical power output can be detected by means of the detector unit 30, which generates corresponding output signals.

The output signals generated by the light emitters 20 and, ultimately, by the detector unit 30 inherently comprise information of movement of the skin 53 as an external object. The SMI induced in the light emitters is sensitive to the position and relative changes thereof. These affect how the emitted light and the back-reflected light interfere.

Due to the pulse caused by moving blood vessels the output signals of the two light emitters 20 have different signal components, due to different relative distances between the skin 53 and the first of the two light emitters and between the skin 53 and the second of the two light emitters (indicated by dashed lines 55 in the drawing). This can be considered the heart rate signal due to pulse.

The distance between the reflecting/scattering surface, i.e. the skin 53, and the light emitters 20 is one parameter which affects interference. In an ideal case, both light emitters have the same distance with respect to the skin and, thus, blood vessels. In this case, differences in signal components in the light emitters provide a measure of pulse and heart rate.

However, unwanted motion of the body part (wrist), skin or tissue, relative to the sensor module results in noise superimposed on the SMI output signals. This is indicated by the arrows 54 in the drawing. The displacement due to such movement, however, can be assumed to affect the output signals of the two light emitters in the same way, i.e. are common mode to both light emitters. However, as discussed above with respect to the distance between the light emitters 20, this noise component may be set to enable a noise component to become common mode and a signal component to be differential mode in the corresponding output signals. Thus, generating a difference signal from the output signals cuts down the motion artifacts significantly while reducing the signal only slightly. The signal-to-noise ratio can be improved significantly.

The process of generating the difference signal is executed by means of the electronic processing unit 40. The electronic processing unit 40 receives the output signals from the light emitters as detected by the detector unit 30. These output signals undergo FFT to yield intermediate signals. These intermediate signals are either subtracted from each other in whole or in part (e.g. only dominating frequencies) to yield a difference signal. As the noise, by placement of the light emitters in the distance described above, is common mode to both light emitters, they tend to cancel in the difference signals. At the same time, the actual signal components are not or only slightly reduced.

The processing unit then receives the difference signals from the sensor module and determines a movement of the skin. The processing unit generates a processed output signal that comprises information of the determined movement to deduce a heart rate.

FIG. 2 shows another exemplary embodiment of a self-mixing interferometry sensor module for a wearable electronic device. The drawing shows a wearable electronic device, e.g. a smartwatch, comprising a sensor module 10, similar to that of FIG. 1. Unlike in the embodiment of FIG. 1, the light emitters are separated by a distance with respect to each other and are arranged with a common tilting angle with respect to the support surface. Thus, the light emitters by design have a different distance with respect to the skin (and the support surface for that matter). This arrangement is suitable for blood flow measurement, for example.

In operation, the wearable electronic device, including the sensor module 10, rests on the skin 53 with the support surface facing down. A difference in signal strength of the output signals may be due to different reflection or scattering at the blood vessel as the external object. Due to the tilted arrangement, a light path may be extended or shortened, depending on the position of a blood vessel 60. Thus, the output signals of the light emitters differ depending on the position of the moving blood vessel. Recording the output signals as a function of time allows to determine a blood flow.

A movement of the sensor module 10, including the rigidly coupled light emitters 20, with respect to the skin 53 affects both light emitters in a similar, or the same, way. Thus, noise due to this movement becomes common mode, similar to the example of FIG. 1. Using essentially the same method and procedural steps, the electronic processing unit 40 receives the output signals from the light emitters 20 as detected by the detector unit 30. These output signals undergo FFT to yield intermediate signals. These intermediate signals are subtracted from each other either in whole or in part (e.g. only dominating frequencies) to yield a difference signal. As the noise is common mode to both light emitters, they tend to cancel in the difference signals. At the same time, the actual signal components are not or only slightly reduced. This allows for increasing spatial resolution further, and signal differences may be more pronounced while noise remains at the same common mode level.

FIGS. 3A, 3B show exemplary measurements conducted with a self-mixing interferometry sensor module for a wearable electronic device. The drawings show an SMI fringe signal (see graphs (a), output intensity vs. time) and their Fourier spectra (see graphs (b) spectra intensity vs. frequency). For example, the data depicted in FIG. 3A have been recorded by a first light emitter 20 and the data depicted in FIG. 3B have been recorded by a second light emitter 20, which are separated from each other by a distance.

The graph in FIG. 3A is dominated by noise motion, which is apparent as a peak frequency at 7 kHz (see dashed line in graph (b)). This peak is proportional to the noise motion velocity. The graph in FIG. 3B shows contributions from both signal and noise motion, which is apparent as a peak frequency at 10 kHz (see dashed line in graph (b)). The difference signal can be determined from the peak difference (e. g. magnitude of peaks), which in this example amounts to 3 kHz. This difference is proportional to motion velocity and has reduced noise contribution.

While this specification contains many specifics, these should not be construed as limitations on the scope of the invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous.

This patent application claims the priority of US patent application 63/344,873, the disclosure content of which is hereby incorporated by reference.

### REFERENCES

10 sensor module
20 light emitters
21 cavity
22 top surface
23 direction of emission
30 detector unit
40 electronic processing unit
50 housing
51 support surface
52 apertures
53 skin (external object)
54 arrow
55 dashed line
60 blood vessel
61 cross-section

1. A self-mixing interferometry sensor module, comprising at least two light emitters of the same type, a detector unit and an electronic processing unit, wherein each light emitter is operable to:
emit coherent electromagnetic radiation out of the sensor module; and
undergo self-mixing interference (SMI) caused by reflections of the emitted electromagnetic radiation from an external object outside the sensor module;
wherein the detector unit is operable to:
generate output signals indicative of the SMI of the light emitters, respectively;
wherein the electronic processing unit is operable to:
generate a difference signal from the output signals indicative of a movement of the external object,
wherein the light emitters are arranged in pairs at a distance from each other, and the distance is set to enable a noise component to become common mode and a signal component to be differential mode in the corresponding output signals.

2. The sensor module according to claim 1, wherein the light emitters have the same configuration, including the same emission wavelengths.

3. The sensor module according to claim 1, wherein the light emitters are rigidly coupled to each other.

4. The sensor module according to claim 1, wherein
the distance is chosen such that the output signals indicative of the SMI of the pair of light emitters differ by a pre-determined amount.

5. The sensor module according to claim 1, comprising an array of the light emitters, wherein the electronic processing unit is operable to generate difference signals from the output signals of pairs of the light emitters.

6. The sensor module according to claim 1, wherein the light emitters comprise:
semiconductor laser diodes, and/or
resonant cavity light emitting devices.

7. The sensor module according to claim 1, wherein the light emitters comprise vertical cavity surface emitting laser diodes.

8. The sensor module according to claim 1, wherein the electronic processing unit is operable to:
conduct a fast Fourier transformation on the output signals to extract a dominant frequency, and
generate the difference signal as a function of the dominant frequency.

9. The sensor module according to claim 1, wherein the light emitters are arranged in parallel such that the light emitters have the same direction of emission.

10. The sensor module according to claim 1, wherein the detector unit is operable to:
detect a junction voltage of the light emitters, respectively, and
generate the output signals as a function of said junction voltages, respectively.

11. The sensor module according to claim 1, wherein the detector unit is operable to:
detect an optical power output of the light emitters, respectively, and
generate the output signals as a function of said optical power outputs, respectively.

12. A wearable electronic device comprising:
a self-mixing interferometry sensor module according to claim 1, and
a housing comprising the sensor module and a support surface to be arranged on the skin of a user, wherein the housing is configured to position the light emitters at a distance from the skin of the user.

13. The wearable electronic device according to claim 12, wherein the light emitters are arranged in the housing, such that the direction of emission of the light emitters is essentially perpendicular or perpendicular to the support surface.

14. The wearable electronic device according to claim 12, wherein the light emitters are arranged in the housing, such that the direction of emission of the light emitters is tilted with respect to the support surface.

15. A method of detecting a movement, comprising the steps of:
emitting, by means of two light emitters of the same type, coherent electromagnetic radiation out of a sensor module,
inducing, within the light emitters, self-mixing interference (SMI) caused by reflections of, or scattering by, the emitted electromagnetic radiation from an object external to the sensor module,
generating output signals indicative of the SMI of the light emitters, respectively, and
generating a difference signal from the output signals indicative of a movement of the external object, wherein
the light emitters are arranged in pairs at a distance from each other, and the distance is set to enable a noise component to become common mode and a signal component to be differential mode in the corresponding output signals.
